(19) United States
(12) Patent Application Publication
(10) Pub. No.: US 2011/0217241 A1
(43) Pub. Date: Sep. 8, 2011

(54) CONJUGATES OF 19F MR IMAGING TRACERS FOR USE IN MULTI-CHROMIC MRI IMAGING

(75) Inventors: Yihua Bruce Yu, Ellicott City, MD (US); Zhong-Xing Jiang, Cockeysville, MD (US)

(73) Assignee: University of Maryland, Baltimore, Baltimore, MD (US)

(21) Appl. No.: 13/128,578

(22) PCT Filed: Nov. 5, 2009
(86) PCT No.: PCT/US09/63381

§ 371 Date: May 10, 2011

Related U.S. Application Data
(62) Division of application No. 61/114,725, filed on Nov. 14, 2008.

(51) Int. Cl. A61K 49/06; C07F 5/00; C07F 1/08; C07F 15/04; C07F 15/02; C07C 235/08; C07K 5/08; C07H 21/04; C12N 5/071

(52) U.S. Cl. 424/9.3; 534/15; 540/465; 564/153; 530/331; 536/23.1; 435/325

(57) ABSTRACT

Compositions for producing multi-chromic (“color”) magnetic resonance images (MRI). The compositions for use as MRI imaging agents include a magnetic resonance imaging tracer having a signal emitter, such as a ¹⁹F imaging tracer (¹⁹FIT), conjugated with a magnetic signal modulator, such as a paramagnetic functional group or a paramagnetic ion. The ¹⁹FIT and the signal modulator (M) are joined covalently by a chelator such as DOTA, forming a nanometer-sized fluorinated paramagnetic complex ¹⁹FIT-DOTA-M. Other signal emitters, such as ³¹P imaging tracer (³¹PIT), can also be modulated using this strategy (i.e., ³¹PIT-DOTA-M) to generate multi-chromic ³¹P MRI or MRS.

### FIELD OF THE INVENTION

This invention relates generally to compositions for use in magnetic resonance imaging (MRI) and magnetic resonance spectroscopy (MRS), and more particularly to paramagnetic compositions with different ¹⁹F magnetic resonance frequencies to be used as imaging agents for multi-chromic (also called multi-spectral) ¹⁹F MRI and ¹⁹F MRS.

### BACKGROUND OF THE INVENTION

Magnetic resonance imaging (MRI) has made great impact on medical diagnosis. MRI is a known technique for obtaining images of the inside of an object under investigation, such as a patient. MRI techniques include the detection of particular atomic nuclei (e.g., those possessing magnetic dipole moments) utilizing magnetic fields and radio-frequency radiation. The hydrogen atom, having a nucleus consisting of a single unpaired proton, has the strongest magnetic dipole moments of nuclei found in biological tissues. As hydrogen occurs in both water and lipids, it is abundant in the human body. Therefore, MRI is most commonly used to produce images based upon the distribution density of protons and/or the relaxation times of protons in organs and tissues. Other nuclei having a net magnetic dipole moment also exhibit a nuclear magnetic resonance phenomenon which may be used in MRI applications. Such nuclei include carbon-13 (six protons and seven neutrons), fluorine-19 (9 protons and 10 neutrons), sodium-23 (11 protons and 12 neutrons), and phosphorus-31 (15 protons and 16 neutrons).

In MRI, the nuclei under study in a sample (e.g., ¹⁹F, etc.) are irradiated with the appropriate radio-frequency (RF) energy in a controlled gradient magnetic field. These nuclei, as they relax, subsequently emit RF energy at a sharp resonance frequency. The resonance frequency of the nuclei depends on the applied magnetic field. In some cases, the concentration of nuclei to be measured is not sufficiently high to produce a detectable magnetic resonance signal. Signal sensitivity may be improved by administering higher concentrations of the target nuclei or by coupling the nuclei to a suitable “probe” which will concentrate in the body tissues of interest.

Diagnostic MRI relies on the ¹H₂O signal, which is suited for providing body information (anatomy and physiology). To apply MRI to the therapeutic arena (therapeutic MRI), it was necessary to develop a new MRI mode which lacks background signal interference. Because there are no endogenous fluorine compounds, ¹⁹F imaging agents can be used to label therapeutic agents (drugs, cells, etc.) so that the therapeutic agents can be tracked and quantified by ¹⁹F MRI and ¹⁹F MRS.

Conventional MRI technologies are mono-chromic in the sense that the signal emitter (¹H or ¹⁹F nucleus) emits signal at only one frequency. With fixed signal frequency, the only variable parameter in conventional MRI is signal intensity, whose variation creates different shades of black and white contrasts, forming the image. Lately, there have been efforts to create multi-chromic ¹H MRI, using either chemical-exchange saturation technique (Aime, S. et al., Angew. Chem. Int. Ed. 44, 1813, 2005) or micro-fabrication technique (Zabow, G. et al., Nature, 453, 1058, 2008). However, both techniques are at their infancy. Furthermore, there has been no report on multi-chromic ¹⁹F MRI. In the context of MRI, multi-chromicity, or “color”, refers to multiple magnetic resonance frequencies, which are actually in the radiofrequency range, rather than in the visible light range.

Mono-chromicity severely limits the application of ¹⁹F MRI as a tracer technology. For example, it is impossible to track two objects simultaneously using mono-chromic ¹⁹F MRI technology because it cannot distinguish them. To achieve simultaneous tracking of multiple objects, it is necessary to develop multi-chromic ¹⁹F imaging agents so that different objects can be labeled by agents with different ¹⁹F resonance frequencies.

There is an ongoing need for improved MRI imaging agents to expand the use of MRI, such as through multi-chromic or color MRI.

### SUMMARY OF THE INVENTION

A general object of the invention is to provide a method for providing multi-chromic (“color”) MRI images, as well as compositions and compounds for use in implementing the method.

The general object of the invention can be attained, at least in part, through a composition including a magnetic resonance (MR) imaging tracer including a magnetic signal emitter, and conjugated with any magnetic signal modulator (M), such as a paramagnetic functional group and/or a chelate complex. The chelate complex includes a ligand combined with a paramagnetic ion or a diamagnetic ion such as a calcium or magnesium ion. In one exemplary composition, the imaging tracer can be a ¹⁹F imaging tracer (¹⁹FIT), having ¹⁹F as the magnetic signal emitter, and the magnetic signal modulator (M) can be a paramagnetic ion. The imaging tracer and the signal modulator are joined covalently by a chelator such as DOTA, forming a nanometer-sized fluorinated paramagnetic complex, ¹⁹FIT-DOTA-M.

When used as imaging agents, the paramagnetic functional group and/or paramagnetic ion act as signal modulators. Different signal modulators result in different magnetic resonance frequencies, and such can be used to provide multi-chromic MR readings when two or more signal modulators are used. The invention further contemplates a method of generating a magnetic resonance image using the compositions of this invention. The method includes administering to an animal a dose of one or more compositions according to this invention and measuring an amount of the composition in a tissue or organ of the animal using magnetic resonance imaging (MRI). Differences in signals from different signal modulators provide for a way to identify unknown metal ions by the resulting resonance shift, as well as to produce multiple varying resonance frequencies.

The modulation of ¹⁹F signal frequency, i.e., the generation of multi-chromicity, can be achieved or customized in any of the following three ways: 1) using different signal modulators (e.g., Fe²⁺, Co²⁺, Ni²⁺, Eu³⁺, Gd³⁺ & Tb³⁺); 2) changing the scaffold of the imaging tracer to adjust the distance between the ¹⁹F nuclei and the paramagnetic ion(s) (M) or changing the number of branches in the scaffold to adjust the molar ratio of ¹⁹F:M; and/or 3) combining complexes generated in 1) and 2) to form supra-molecular paramagnetic complexes. In this last way, charged groups (e.g., —NH₃⁺ and —COO⁻) will be introduced into the scaffold of the ¹⁹F imaging tracer to form positively and negatively charged ¹⁹F imaging tracers (¹⁹FIT⁺ and ¹⁹FIT⁻, respectively), which will then be combined to form supra-molecular paramagnetic complexes (e.g., ¹⁹FIT⁺ plus ¹⁹FIT⁻,) via electrostatic attractions.

### Definitions

As used herein, the term “alkyl” refers to a hydrocarbon group that can be conceptually formed from an alkane, alkene, or alkyne by removing hydrogen from the structure of a cyclic or non-cyclic hydrocarbon compound having straight or branched carbon chains, and replacing the hydrogen atom with another atom or organic or inorganic substituent group. In some aspects of the invention, the alkyl groups are “C₁ to C₆ alkyl” such as methyl, ethyl, propyl, isopropyl, n-butyl, iso-butyl, sec-butyl, tert-butyl, amyl, tert-amyl, and hexyl groups, their alkenyl analogues, their alkynyl analogues, and the like. Many embodiments of the invention comprise “C₁ to C₄ alkyl” groups that include methyl, ethyl, propyl, iso-propyl n-butyl, iso-butyl, sec-butyl, and t-butyl groups, their alkenyl analogues, their alkynyl analogues, or the like. Some of the preferred alkyl groups of the invention have three or more carbon atoms preferably 3 to 16 carbon atoms, 4 to 14 carbon atoms, or 6 to 12 carbon atoms. The alkyl group can be unsubstituted or substituted. A hydrocarbon residue, for example an alkyl group, when described as “substituted,” contains or is substituted with one or more independently selected heteroatoms such as O, S, N, P, or the halogens (fluorine, chlorine, bromine, and iodine), or one or more substituent groups containing heteroatoms (OH, NH₂, NO₂, SO₃H, and the like) over and above the carbon and hydrogen atoms of the substituent residue. Substituted hydrocarbon residues may also contain carbonyl groups, amino groups, hydroxyl groups and the like, or contain heteroatoms inserted into the “backbone” of the hydrocarbon residue. In one aspect, an “alkyl” group can be fluorine substituted. In a further aspect, an “alkyl” group can be perfluorinated.

In certain aspects, the term “alkyl” as used herein is a branched or unbranched saturated hydrocarbon group of 1 to 24 carbon atoms, for example 1 to 12 carbon atoms or 1 to 6 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, substituted or unsubstituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein. A “lower alkyl” group is an alkyl group containing from one to six carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The terms “amine” or “amino” as used herein are represented by the formula NA¹A²A³, where A¹, A², and A³ can be, independently, hydrogen or substituted or unsubstituted alkyl, cycloalkyl, alkenyl, cycloalkenyl, alkynyl, cycloalkynyl, aryl, or heteroaryl group as described herein.

The term “carboxylic acid” or “carboxyl group” as used herein is represented by the formula —C(O)OH.

The term “ester” as used herein is represented by the formula —OC(O)A¹ or —C(O)OA¹, where A¹ can be a substituted or unsubstituted alkyl, cycloalkyl, alkenyl, cycloalkenyl, alkynyl, cycloalkynyl, aryl, or heteroaryl group as described herein.

The term “hydroxyl” as used herein is represented by the formula —OH.

The term “silyl” as used herein is represented by the formula —SiA¹A²A³, where A¹, A², and A³ can be, independently, hydrogen or a substituted or unsubstituted alkyl, cycloalkyl, alkoxy, alkenyl, cycloalkenyl, alkynyl, cycloalkynyl, aryl, or heteroaryl group as described herein.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture.

Other objects and advantages will be apparent to those skilled in the art from the following detailed description taken in conjunction with the appended claims.

### DESCRIPTION OF THE DRAWINGS

FIGS. 1-3 illustrate representative compositions according to embodiments of this invention.

FIG. 4 illustrates the use of compositions according to this invention as labels for different therapeutic or diagnostic objects.

FIG. 5 illustrates the use of compositions according to this invention for identifying and removing undesirable metal ions (stable or radioactive) from a mammal or other environment.

FIG. 6 is a graph illustrating the structure and chemical shift of an exemplary ¹⁹F imaging tracer using different signal modulators according to one embodiment of this invention.

### DETAILED DESCRIPTION OF EMBODIMENTS OF THE INVENTION

The present invention provides compositions for use in magnetic resonance imaging (MRI). The compositions use paramagnetic complexes providing different magnetic resonance frequencies for use as imaging agents for multi-chromic (also called multi-spectral) MRI. To achieve multi-chromic MRI, the resonance frequency of an imaging agent needs to be shifted discretely and in a controllable manner. Resonance frequencies of nuclear spins are shifted according to this invention by paramagnetic groups or ions, whose electronic magnetic moment, μₑ, will exert an effect on the nuclear magnetic moment (spin). Paramagnetic ions (with unpaired electrons) cause much greater frequency shift than diamagnetic ions (no unpaired electrons).

Paramagnetic ions also reduce the relaxation times (T₁ and T₂) of ¹⁹F through paramagnetic relaxation enhancement (Belle, C. et al., Coord. Chem. Rev. asap format, 2008 (DOI: 10.1016/j.ccr.2008.06.015)). Reduction of T₁ leads to increased signal intensity, though T₂ is also reduced, requiring identification of paramagnetic complexes that effectively reduce T₁ without over-reducing T₂. Hence, paramagnetic ions will shift signal frequency (allowing for the creation of “color”) and potentially enhance signal intensity (raising sensitivity) simultaneously, playing a dual beneficial role for multi-chromic ¹⁹F MRI.

Before the present compounds, compositions, and/or methods are disclosed and described, it is to be understood that they are not limited to specific imaging agents or synthetic methods unless otherwise so explicitly stated, or to particular reagents unless so otherwise explicitly stated, as such may, of course, vary. It is also to be understood that the terminology used herein is for the purpose of describing particular embodiments only and is not intended to be limiting.

In one embodiment of this invention, the composition is an imaging agent comprising a magnetic resonance (MR) imaging tracer, which contains one or more magnetic signal emitters, conjugated with a magnetic signal modulator. The composition and the use thereof as an imaging tracer in the methods of this invention are not limited to the use of any particular imaging tracer. Various and alternative known and available MRI imaging tracers, including both positive and negative imaging tracers, are suitable for use in the imaging agent and methods of this invention. Exemplary imaging tracers include, without limitation, compounds containing as their active signal emitter element fluorine, phosphorus, gadolinium, manganese, and/or iron.

Imaging agents of this invention use signal modulators for shifting the magnetic resonance of the signal emitter (e.g., ¹⁹F or ³¹P). Exemplary signal modulators for use with the compositions of this invention include paramagnetic materials, such as paramagnetic functional groups and paramagnetic ions. One such paramagnetic functional group is nitroxide. Paramagnetic ions can be conjugated into the compositions of this invention in a chelate complex by a ligand. Exemplary paramagnetic ions include monovalent, divalent, and trivalent metal ions such as Cu²⁺, Ni²⁺, Fe³⁺, Eu³⁺, Gd³⁺, Tb³⁺, and combinations thereof. Any suitable ligand for forming a chelate complex with a paramagnetic ion is available for use in the composition of this invention. One exemplary ligand is 1,4,7,10-tetraazacyclododecane-1,4,7,10-tetraacetic acid (DOTA) or derivatives thereof.

The composition can include one type of signal modulator or more than one type of signal modulator, thereby providing further possibilities of resonance shifts. Also, two compositions having two different signal modulators can be desirably used together to provide multi-chromic images. Two differing signal modulations can be obtained as described above, for example, by using a first imaging tracer including a first paramagnetic functional group and/or chelate complex, and a second same or different imaging tracer including a second paramagnetic functional group and/or a chelate complex.

In one particularly preferred embodiment of this invention, a suitable imaging tracer is or includes a fluorocarbon. A desirable fluorocarbon imaging tracer includes at least one, and preferably a plurality of, flourine-19 (¹⁹F) nuclei, which are detectable by ¹⁹F MRI and ¹⁹F MRS. Naturally occurring fluorine nuclei (¹⁹F) generally provide a clear nuclear magnetic resonance signal, and thus can function as imaging agents in MRI and MRS. Particular benefits of using ¹⁹F include: 1) an extremely low and undetectable endogenous concentration in the body (fluorine is not naturally found in the body), 2) a high nuclear magnetic resonance sensitivity, and 3) a magnetogyric ratio close to that of ¹H, thus permitting ¹⁹F magnetic resonance imaging to be carried out with only minor modifications of existing MRI equipment.

In one embodiment of this invention the imaging tracer for use in conjugation to form the composition of this invention comprises a compound including the structure:

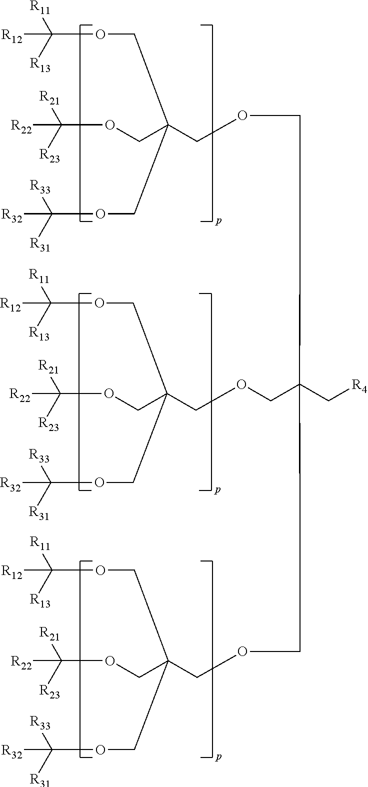

where p is a non-negative integer; R₁₁, R₁₂, R₁₃, R₂₁, R₂₂, R₂₃, R₃₁, R₃₂, and R₃₃ are independently, H, CH₃, CF₃, or alkyl; and R₄ is H, OH, OBn, OC(CF₃)₃, alkyl, or alkoxy. In a further embodiment, p is 2, 3, 4, or 5. In one embodiment, at least one of R₁₁, R₁₂, R₁₃, R₂₁, R₂₂, R₂₃, R₃₁, R₃₂, or R₃₃ is CF₃. In a further embodiment, R₁₁, R₁₂, R₁₃, R₂₁, R₂₂, R₂₃, R₃₁, R₃₂, and R₃₃ are CF₃.

In a yet further embodiment, the imaging tracer comprises a compound including the structure:

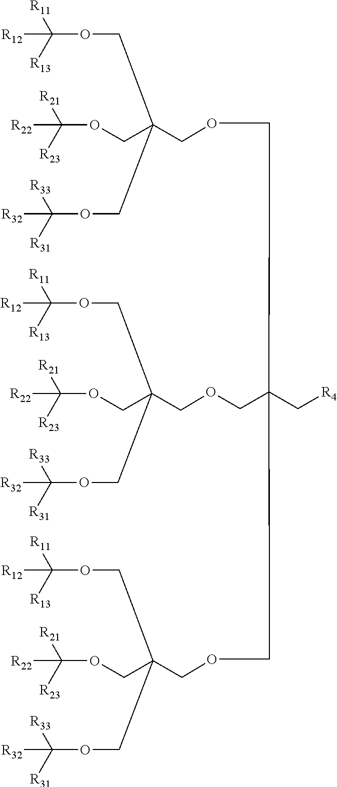

where R₁₁, R₁₂, R₁₃, R₂₁, R₂₂, R₂₃, R₃₁, R₃₂, and R₃₃ are, independently, H, CH₃, CF₃, or alkyl; and R₄ is H, OH, OBn, OC(CF₃)₃, alkyl, or alkoxy. In one aspect, at least one of R₁₁, R₁₂, R₁₃, R₂₁, R₂₂, R₂₃, R₃₁, R₃₂, or R₃₃ is CF₃. In a further aspect, R₁₁, R₁₂, R₁₃, R₂₁, R₂₂, R₂₃, R₃₁, R₃₂, and R₃₃ are CF₃.

One particularly preferred imaging tracer comprises the structure:

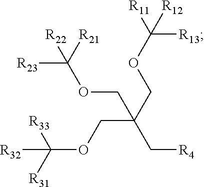

where R₁₁, R₁₂, R₁₃, R₂₁, R₂₂, R₂₃, R₃₁, R₃₂, and R₃₃ are, independently, H, CH₃, CF₃, or alkyl, and R₄ is H, OH, OBn, OC(CF₃)₃, alkyl, or alkoxy. In a further embodiment, R₁₁, R₁₂, and R₁₃ are CF₃. In a further embodiment, R₂₁, R₂₂, and R₂₃ are CF₃. In a further embodiment, R₃₁, R₃₂, and R₃₃ are CF₃. In one embodiment, at least one of R₁₁, R₁₂, R₁₃, R₂₁, R₂₂, R₂₃, R₃₁, R₃₂, or R₃₃ is CF₃. In a further aspect, R₁₁, R₁₂, R₁₃, R₂₁, R₂₂, R₂₃, R₃₁, R₃₂, and R₃₃ are CF₃.

The composition of this invention including the imaging tracers described above is desirably conjugated at or through the R₄ position to a paramagnetic functional group or a chelate complex. The paramagnetic functional group or chelate complex conjugated to the imaging tracer at or through the R₄ position is not intended to be limited to any particular paramagnetic functional group or chelate complex.

In one embodiment of this invention, the composition includes an MR imaging tracer that is conjugated to a plurality of signal modulating paramagnetic functional groups and/or chelate complexes. The MR imaging tracer includes at the R₄ position, a branching module including a plurality of branching units. Each of the branching units is conjugated to one of the plurality of signal modulators. An exemplary branching module comprises iminodicarboxylic acid.

In one embodiment of this invention, R₄ of any of the above imaging tracers comprises the structure:

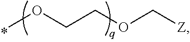

where q is a non-negative integer (such as 0-3), and Z comprises a paramagnetic functional group, a chelate complex or a substituted or unsubstituted amide. One exemplary substituted or unsubstituted amide comprises the structure:

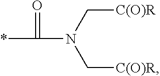

where each R is OH, NH₂, NH-alkyl, alkyl, a polyalkylene oxide, or further conjugated to the paramagnetic functional group or the chelate complex. In another embodiment, the substituted or unsubstituted amide comprises the iminocarboxylic acid structure:

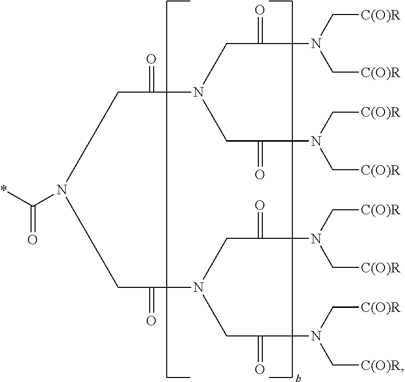

where b is a non-negative integer, and each R is OH, NH₂, NH-alkyl, alkyl, a polyalkylene oxide, or further conjugated to the paramagnetic functional group or the chelate complex. In an exemplary embodiment, b is 0, 1, 2, or 3.

The composition of this invention can optionally include a hydrophilicity enhancing module connecting each of the plurality of branching units to the corresponding one of the plurality of signal modulators. Referring to the branching units described above, a hydrophilicity enhancer can be attached at the R position. Desirably, the composition includes a hydrophilicity enhancing module connecting each of the plurality of branching units to one of the plurality of signal modulating paramagnetic functional groups or chelate complexes. Hydrophilicity enhancing modules according to this invention help ensure rapid renal excretion of the imaging tracer, for example, after the imaging agent is cleaved in vivo from the therapeutic agent (discussed further below). An exemplary hydrophilicity enhancer for use according to this invention is an oligo-oxyethylene.

The following structures represent exemplary imaging agents according to one embodiment of this invention, where X is a paramagnetic functional group or a chelate complex according to this invention.

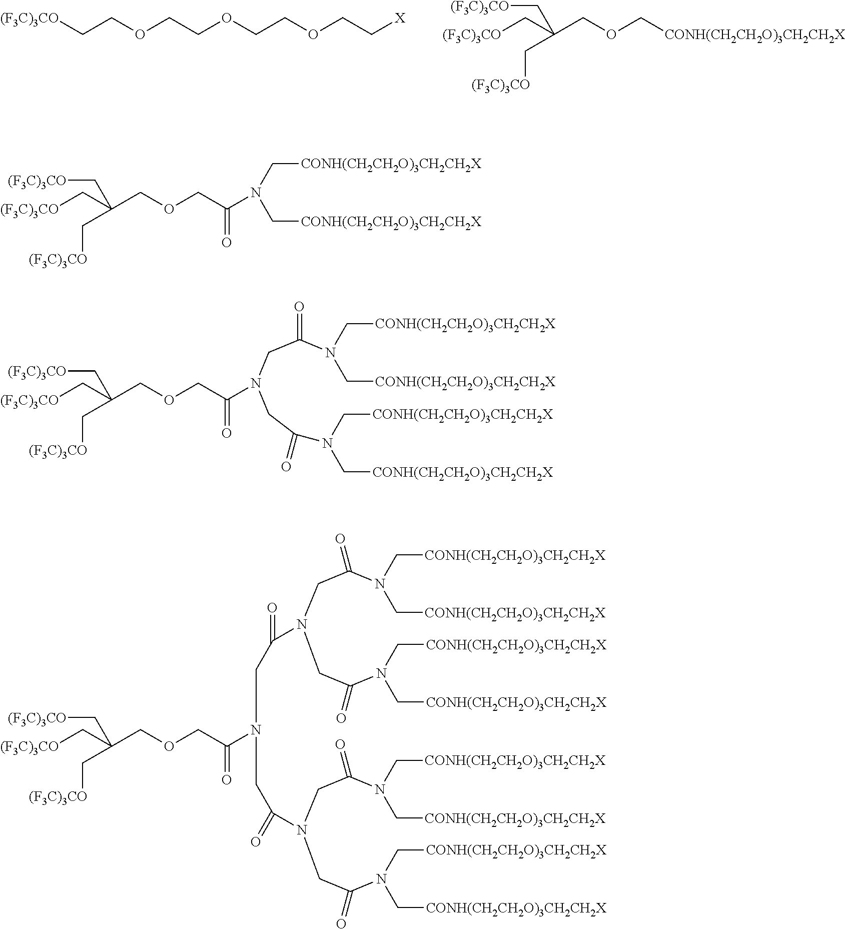

An exemplary process for the preparation of an imaging tracer having the structure:

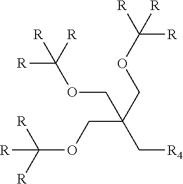

where R is H, CH₃, CF₃, fluorocarbon, or alkyl and wherein R₄ is H, OH, OBn, alkyl, or alkoxy, includes the steps of: providing a triol, and reacting the triol with tert-butanol or nonafluoro-tert-butanol to provide a tri-tert-butyl ether or a triperfluoro-tert-butyl ether. The reacting step can be performed with nonafluoro-tert-butanol. The triol can be pentraerythritol, mono-silylated pentraerythritol, or 2,2-bis-hydroxymethyl-propan-1-ol. The providing step can be performed by the steps of: mono-protecting pentraerythritol before the reacting step, and deprotecting the product of the reacting step. The reacting step can occur before the deprotecting step. Also, the process can further include the step of coupling the product the deprotecting step with a hydrophilic compound, such as a moiety having the structure:

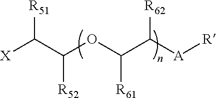

where n is 0 or a positive integer; R₅₁, R₅₂, R₆₁, and R₆₂ are, independently, H or alkyl; R′ comprises H, CH₂CO₂H, silyl, or alkyl; A is O, S, or amino; and X is a leaving group. n can be an integer from 4 to 12. Also, the process can include the step of cleaving the silyl group. The process can further include the step of conjugating with cyclen or a compound comprising a cyclen residue.

The following two Schemes illustrate exemplary reactions to obtain suitable ¹⁹F imaging tracers (including the fluorocarbon module, branching unit, and hydrophilicity enhancer) for further conjugation with a signal modulator according to this invention.

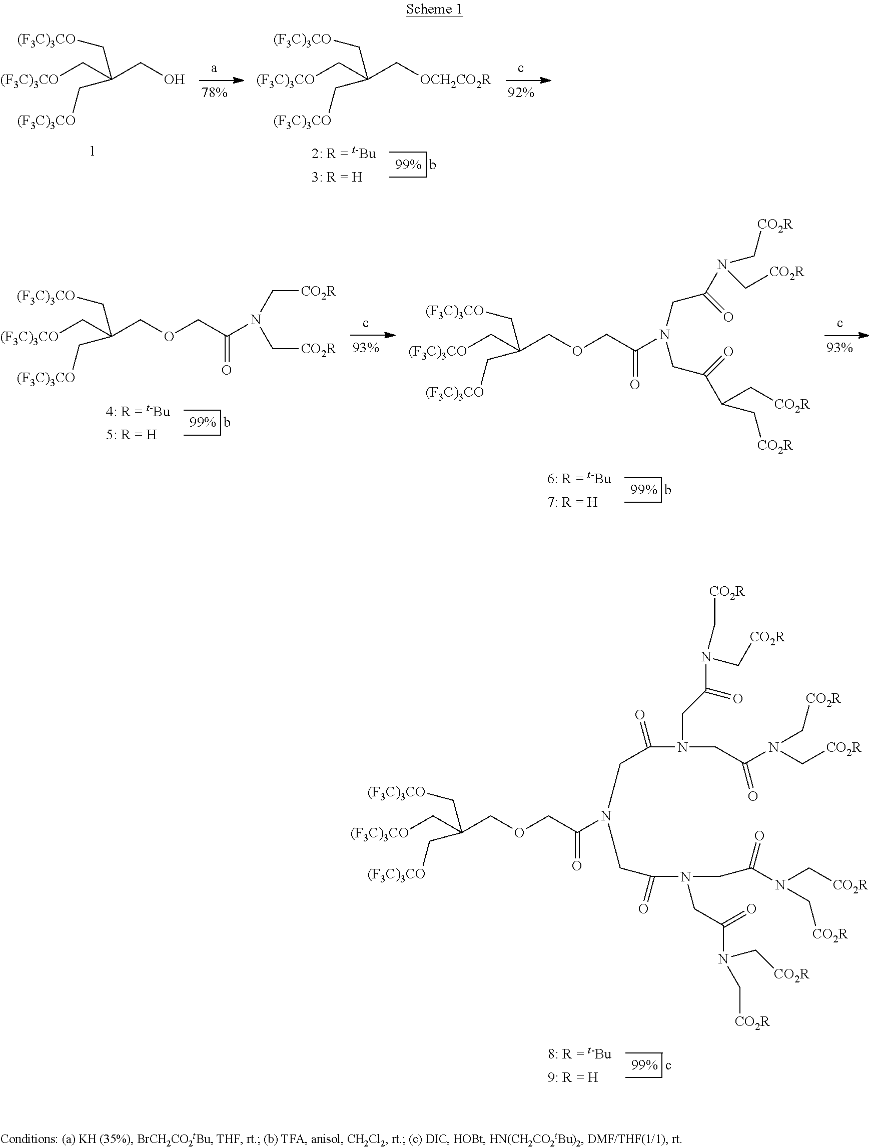

Treatment of alcohol 1 with potassium hydride and tert-butyl bromoacetate gives ester 2 after simple phase separation of the quenched reaction mixture. Ester 2 reacted with trifluoroacetic acid gives the acid 3 after removal of reaction solvent, anisol, and TFA. Acid 3 is coupled with di-tert-butyl iminodiacetate to yield ester 4 after fluorous solid phase extraction. By repeating the coupling and deprotecting processes, the further branched intermediates are obtained.

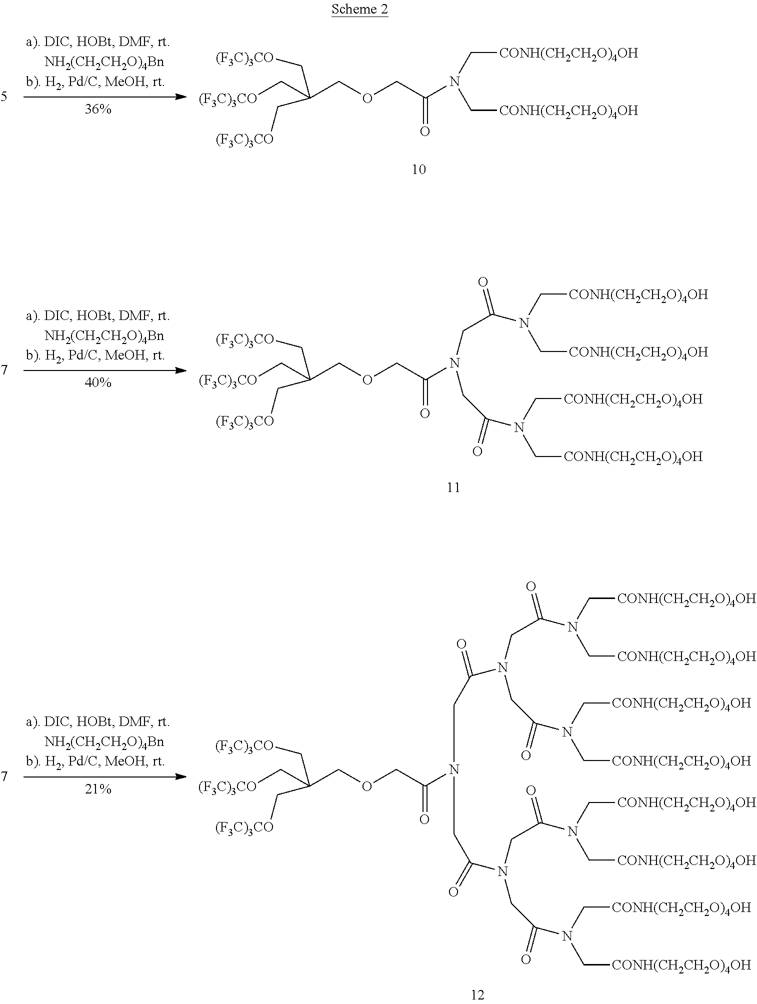

As discussed above, the composition of this invention includes a signal emitter that allows the compositions use as an imaging agent, and a signal modulator. In one embodiment of this invention, the composition has the following structure, which can be made, for example, according to the above Schemes:

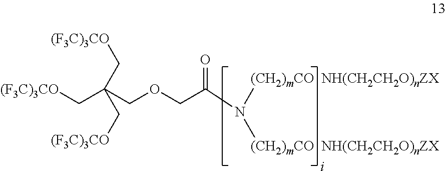

where each of the two or more X groups is either a paramagnetic functional group, a chelate complex or an ¹H contrast agent (in case a dual-nuclei (e.g., ¹H-¹⁹F) imaging agent is desired). At least one X group is preferably a paramagnetic functional group or a chelate complex if signal modulator is desired. Each Z is a linker group, such as a heterocyclic group (for example, a triazole ring), or a linker bond, such as an amide bond or an ester bond. Each variable “m” is independently a positive integer, while each “n” and “i” is independently zero or a positive integer. A further exemplary composition has the following structure:

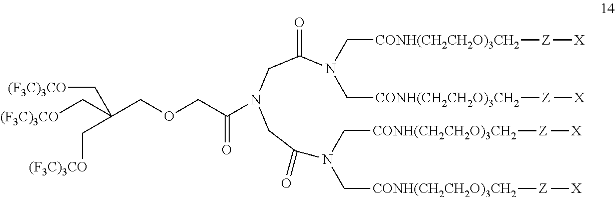

where Z and X are as described above.

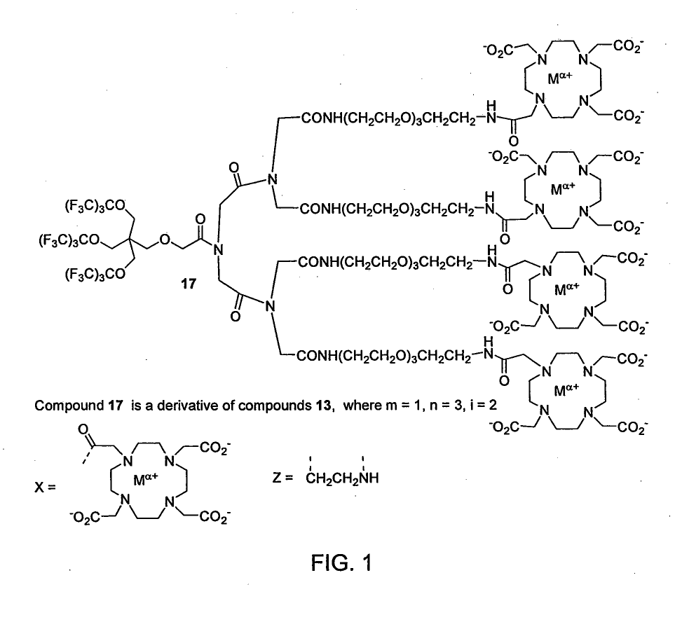

FIG. 1 illustrates the structure of Compound 17, a representative imaging agent of this invention having a ¹⁹F imaging tracer (¹⁹FIT) and a signal modulator M^{α+} joined by the chelator 1,4,7,10-tetraazacyclododecane-1,4,7,10-tetraacetate (DOTA). Compound 17 is one of many useful derivatives of Compounds 13 and 14 above, and the signal modulator M^{α+} can be any of the paramagnetic ions discussed herein. The ¹⁹FIT is a bi-spherical dendrimer which provides the F-sphere that emits a single ¹⁹F signal from (in this example) twenty-seven spherically-symmetric fluorine nuclei. The H-sphere provides anchoring points for the signal modulator. Between the F-sphere and the H-sphere are the hydrophilic segments that act as a structural scaffold and the water-solubility enhancers discussed above. An exemplary synthesis of Compound 17 is illustrated in Scheme 3. In the exemplary Scheme 3, M^{α+} of Compound 17 is Gd³⁺. It is to be appreciated that the reaction schemes illustrated in this disclosure are examples of particular ¹⁹FIT syntheses, and are not intended to be limiting, as not all ¹⁹FIT synthesis will follow that same route as shown.

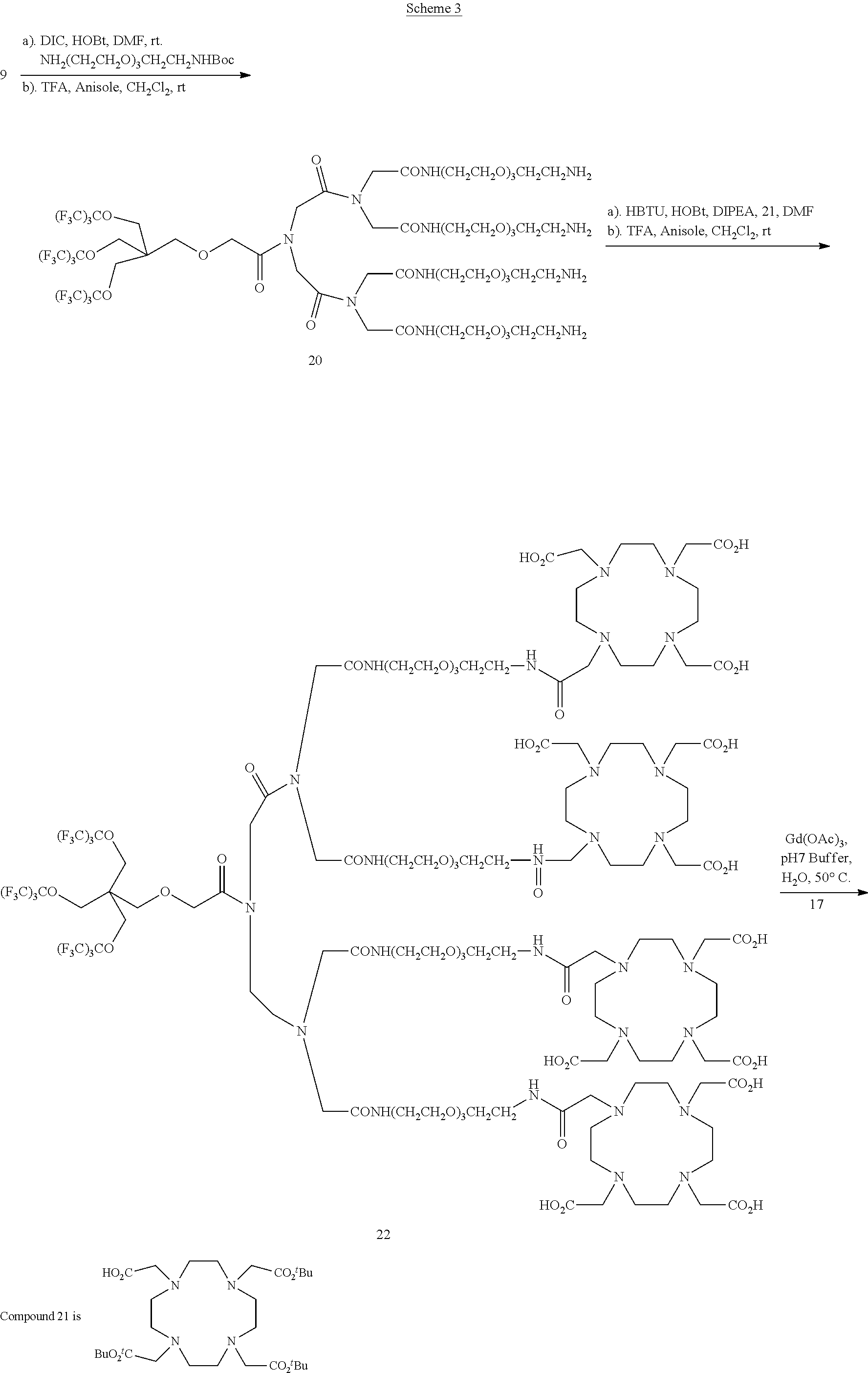

As shown in FIG. 1, parts of ¹⁹FIT-DOTA are joined by peptide bonds. The assembly of the components in one embodiment proceeds sequentially from the F- to the H-sphere and then to DOTA (commercially available in its tris(tBu)-protected form), in a fashion analogous to peptide synthesis. NMR spectroscopy (¹H, ¹³C & ¹⁹F) and mass spectrometry can be used to verify synthesis intermediates and products. All final products and key intermediates can be purified by HPLC. ¹⁹FIT-DOTA is then constituted with the paramagnetic ion M to form the fluorinated paramagnetic complex ¹⁹FIT-DOTA-M.

The modulation of the ¹⁹F signal frequency of the composition of this invention can be modified by using different signal modulators, but also by changing the scaffold of the signal emitter to adjust the distance between the ¹⁹F nuclei and the signal modulator. The number of branches in the scaffold, such as shown above in Scheme 2, can also be changed to adjust the molar ratio of ¹⁹F:M.

The above embodiments place the ¹⁹FIT on one end and the signal modulator on the opposing branched end. However, this structure is not intended to be limiting, as the end components can be switched, such that the ¹⁹FIT is connected to the branched end of the composition. In one embodiment of this invention, the composition comprises the structure:

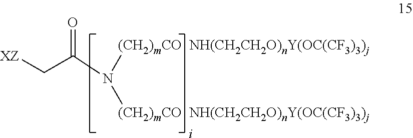

where X includes a paramagnetic functional group, a chelate complex, and/or an ¹H contrast agent. Each Y and Z is independently a linker group or linker bond, each m is independently a positive integer, each j is independently a positive integer, each n is independently zero or a positive integer, and each i is independently zero or a positive integer.

FIG. 2 illustrates the structure of Compound 18, another representative imaging agent of this invention having a ¹⁹F imaging tracer (¹⁹FIT) and a signal modulator M^{α+} joined by the chelator 1,4,7,10-tetraazacyclododecane-1,4,7,10-tetraacetate (DOTA). Compound 18 is one of many useful derivatives of Compound 15 above, where (referring to Compound 15) m=1, n=3, i=2, j=1, Y is CH₂CH₂, Z is an amine linker, and X is DOTA. The signal modulator M^{α+} in Compound 18 can be any of the paramagnetic ions discussed herein. Scheme 4 illustrates an exemplary synthesis of Compound 18. In the exemplary Scheme 4, M^{α+} of Compound 18 is Gd³⁺.

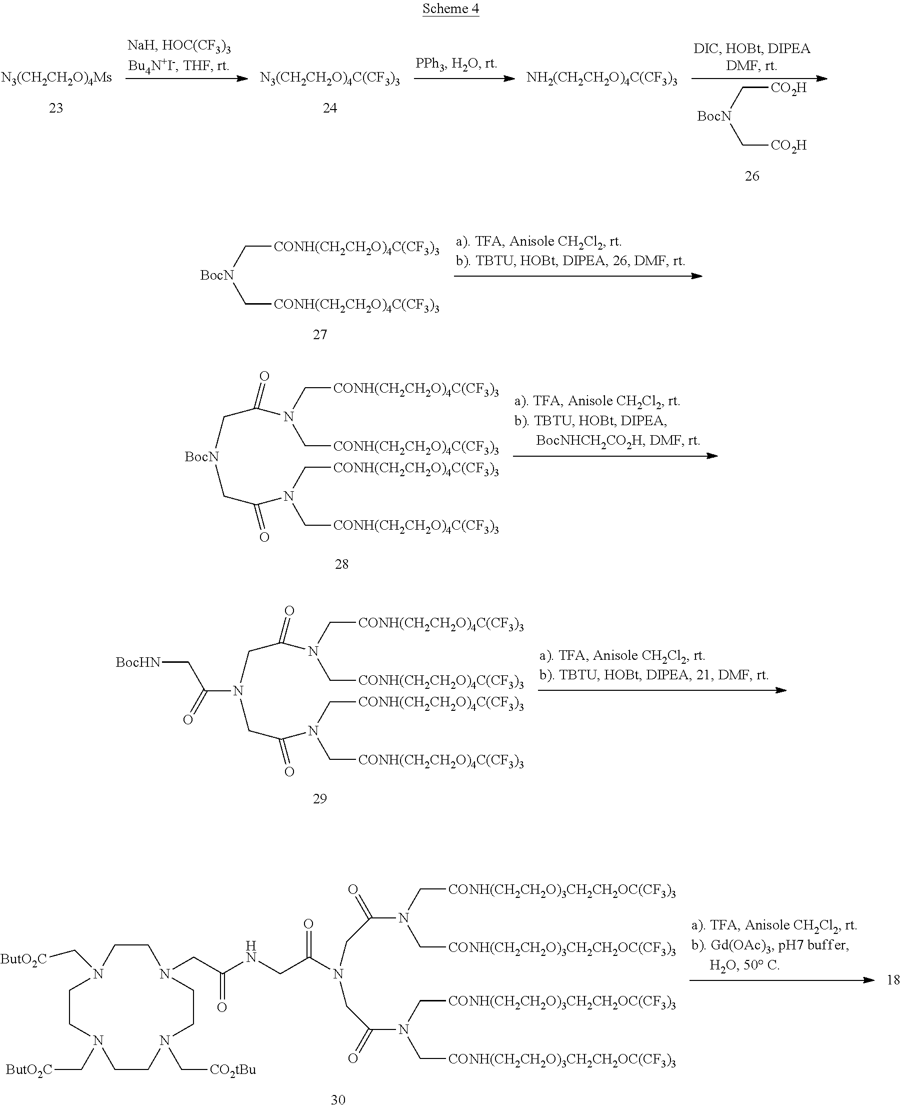

Further variations of the composition are contemplated. In yet another exemplary embodiment of this invention, the composition has the structure:

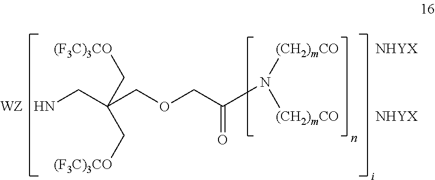

where each X and W is independently includes the paramagnetic functional group, the chelate complex, and/or an ¹H contrast agent. Desirably, at least one X or W includes the paramagnetic functional group or the chelate complex of this invention. Each Y and Z is independently a linker group or bond, such as described above. Each m is independently a positive integer; and n and i are both positive integers.

FIG. 3 illustrates the structure of Compound 19, another representative imaging agent of this invention having a ¹⁹F imaging tracer (¹⁹FIT) and a signal modulator M^{α+} joined by the chelator 1,4,7,10-tetraazacyclododecane-1,4,7,10-tetraacetate (DOTA). Compound 19 is one of many useful derivatives of Compound 16 above, where (referring to Compound 16) m=1, n=2, i=1, W is the DOTA chelate complex, Y is (CH₂CH₂O)₄, Z is an amide linker, and X is hydrogen. Scheme 5 illustrates an exemplary synthesis of Compound 19. In the exemplary Scheme 5, M^{α+} of Compound 19 is Gd³⁺.

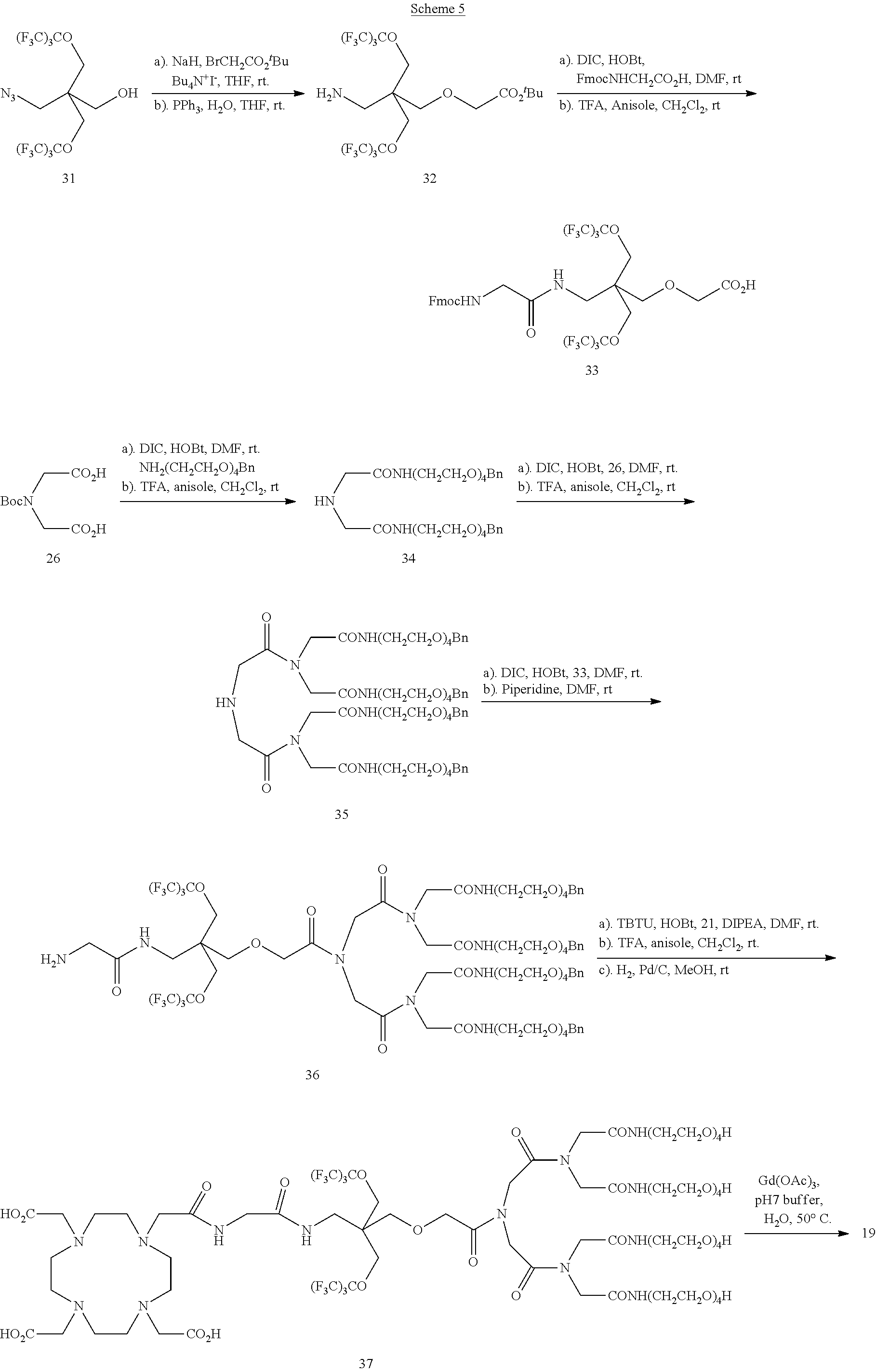

The compositions of this invention, such as ¹⁹FIT-DOTA-M, are nanometer-sized objects. This is in sharp contrast to micrometer-sized particles for multi-chromic ¹H MRI (Zabow, G. et al., Nature, 453, 1058, 2008). As nanometer-sized materials, the compositions of this invention have much wider applicability in biotechnology and biomedicine. In one embodiment of this invention a therapeutic agent is joined to the composition. Exemplary therapeutic agents include drugs, prodrugs, genes, cells, and implants. By labeling drugs, cells, genes, and implants with paramagnetic complexes of different ¹⁹F resonance frequencies, simultaneously tracking of these different objects can be done non-invasively using ¹⁹F MRI by using a different signal modulator for each object to be tracked. For example, using ¹⁹F MRI to track stem cells is an emerging research topic (Bulte, J. W. Nat. Biotech. 23, 945, 2005).

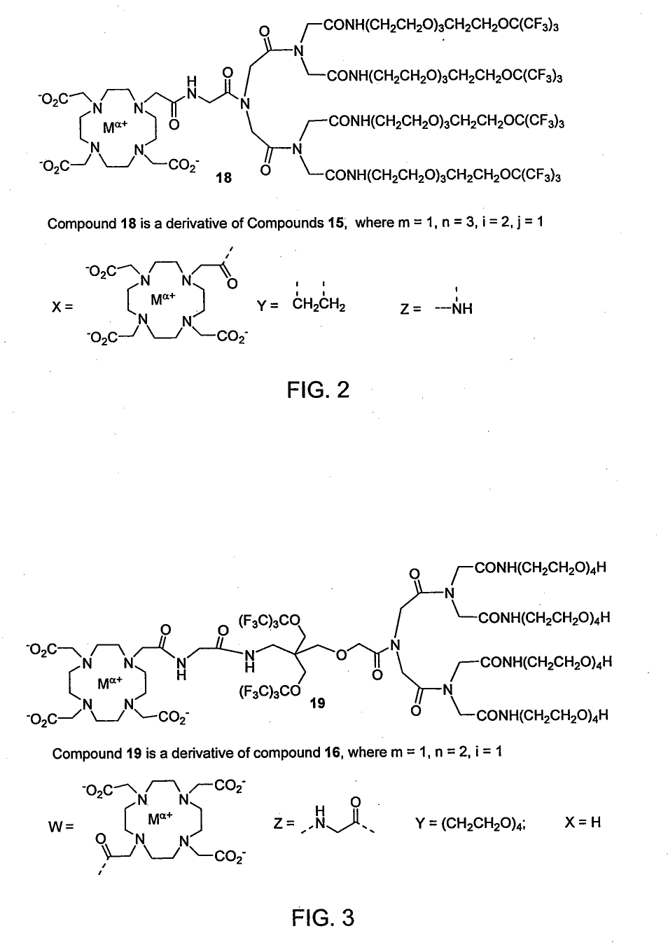

FIG. 4 generally illustrates the labeling of various therapeutic agents with ¹⁹ FIT compositions of this invention. Using FIT with different signal modifiers (e.g., paramagnetic ions i-vii in FIG. 4) as labels results in different detectable wavelengths in the radio-frequency range (i.e., different “colors”), which allows for tracking of the coupled therapeutic agent in vivo. As each of the eight therapeutic agents of FIG. 4 is coupled to a different wavelength emitting ¹⁹FIT (the colors represented by the different crosshatching in the figures being arbitrarily chosen for purposes of explanation), all eight therapeutic agents can be tracked simultaneously in a non-invasive manner using the hetero-nuclear color MRI according to this invention.

To gain wide application in biomedicine, object labeling procedures should be facile and robust. The overall strategy of one embodiment of this invention is to provide a modular design for the imaging agent. Each functional module will be prefabricated and desirably needs no protection during the labeling process. The labeling reactions are desirably reactions that can be carried out in aqueous solutions, such as using thiol-ether formation.

Since cells are much larger than the ¹⁹FIT/signal modulator complexes (¹⁹FIT-M) of this invention, labeling cells with chemically inert ¹⁹FIT-M is unlikely to significantly impact their bioactivities. Compared with the labeling of cells, genes and implants, the labeling of drugs can be the most challenging as most drug molecules are smaller than ¹⁹FIT-M. Therefore, labeling drugs with ¹⁹FIT-M may affect their bioactivities. In one embodiment of this invention, the labeled drug (¹⁹FIT-M-drug) is a prodrug that is pharmacologically inactive but will be converted to the active free drug at the pathological site. One exemplary prodrug, capecitabine (CAP) is the prodrug of the anti-metabolite 5-fluorouracil (5-FU). CAP (Xeloda®) is used for treating colorectal and breast cancers and is converted to 5-FU preferentially in tumor tissues. CAP is enzymatically converted to its active cancer drug form 5-FU, in three steps (CAP→5′-DFCR→5′-DFUR→5-FU), as shown below, with the last step catalyzed by thymidine phosphorylase (TP), preferably within a tumor.

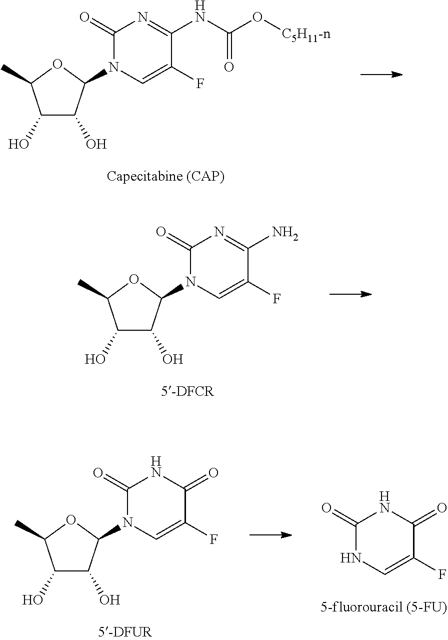

¹⁹FIT-M can be conjugated to the pro-moiety of CAP to form the new prodrug ¹⁹FIT-M-CAP. In vivo conversion of ¹⁹FIT-M-CAP to 5-FU can be monitored by ¹⁹F magnetic resonance spectroscopy (¹⁹F MRS). Also, a long flexible spacer between ¹⁹FIT-M and the prodrug can be used to reduce or eliminate issues of the bulky ¹⁹FIT-M blocking the enzyme-catalyzed prodrug to drug conversion. Scheme 6 illustrates an exemplary conjugation of the CAP with ¹⁹FIT-DOTA-M. ¹⁹FIT-M can be conjugated in similar fashion to 5′-DFCR and 5′-DFUR to form prodrugs of 5-FU. The ¹⁹FIT-M can be attached to the pro-moiety of existing pro-drugs of other drugs in addition to 5-FU.

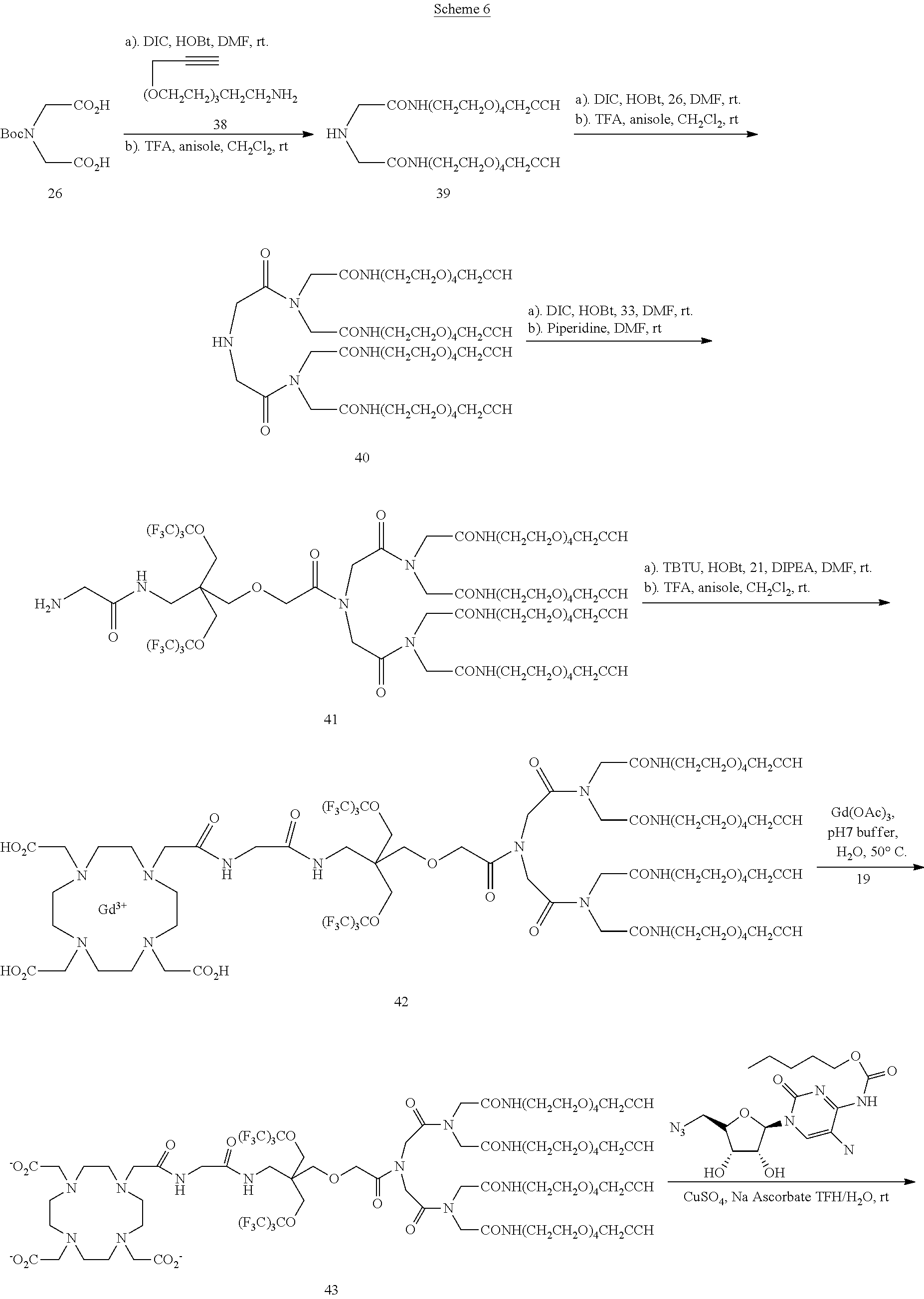

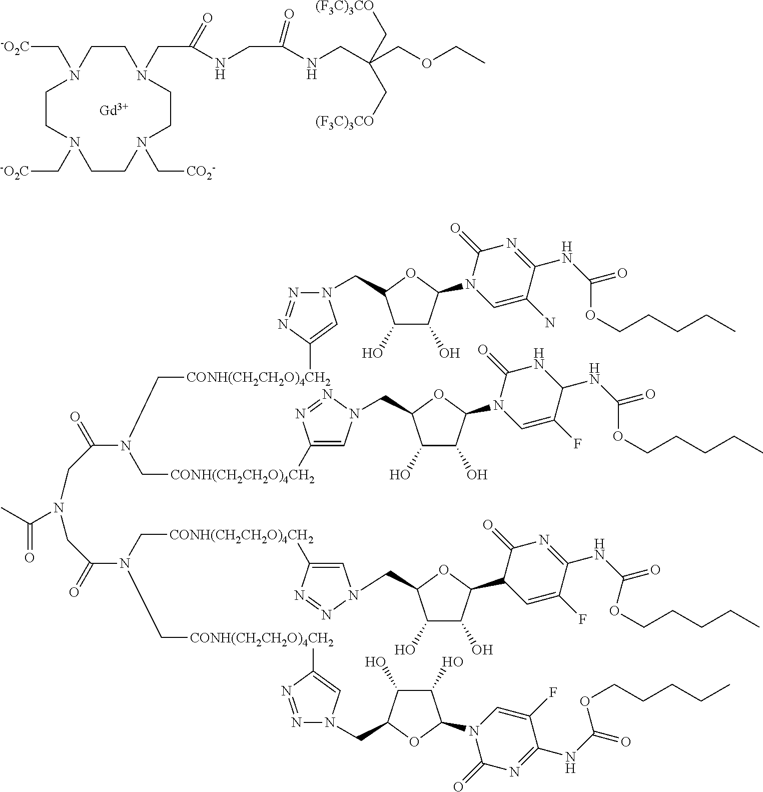

¹⁹F is the second most sensitive nucleus for MR imaging with a sensitivity of 83% of that of ¹H. ¹⁹F imaging is suitable for measuring therapeutic agent concentration in an animal according to this invention because there is no detectable background ¹⁹F signal in animal bodies.

The present invention also provides a pharmaceutical composition comprising a pharmaceutically acceptable carrier and a composition of this invention. Suitable pharmaceutically acceptable carriers described herein, for example, vehicles, adjuvants, excipients, and diluents, are well-known to those skilled in the art and are readily available to the public. The choice of carrier will be determined, in part, by the particular composition and by the particular method used to administer the composition. Accordingly, there are a wide variety of suitable formulations of the pharmaceutical compositions of the present invention.

The present invention also relates to method of using these compositions as imaging agent in combination with treating diseases or conditions, by administering to a subject in need thereof an effective amount of one or more therapeutic agents each joined to an imaging agent in accordance with the present invention. The imaging agents of this invention can be used to monitor or track the dispersion of the therapeutic agents in the body to help ensure effective treatment and effective dosing. The imaging agents also allow for the measurement of amounts of therapeutic agents in a particular area of the body, which can be used to avoid overdosing. The term “treating” is used conventionally, e.g., the management or care of a subject for the purpose of combating, alleviating, reducing, relieving, improving, etc., one or more of the symptoms associated with the disease. The treatment can be prophylactic or therapeutic. “Prophylactic” refers to any degree in inhibition of the onset of a cellular disorder, including complete inhibition, such as in a patient expected to soon exhibit the cellular disorder. “Therapeutic” refers to any degree in inhibition or any degree of beneficial effects on the disorder in the mammal (e.g., human), e.g., inhibition of the growth or metastasis of a tumor. The compositions of this invention are also suitable for use in research and development of new therapeutic agents, allowing for measurements of amounts and distribution in the body during testing.

One skilled in the art will appreciate that suitable methods of administering a therapeutic agent conjugated with a composition of this invention to an animal, e.g., a mammal such as a human, are known. Although more than one route can be used to administer a particular composition, a particular route can provide a more immediate and more effective result than another route.

Formulations suitable for oral administration can consist of (a) liquid solutions, such as an effective amount of a therapeutic agent of this invention dissolved in a diluent, such as water or saline, (b) capsules, sachets or tablets, each containing a predetermined amount of the active ingredient, as solids or granules, (c) suspensions in an appropriate liquid, and (d) suitable emulsions.

Tablet forms can include one or more of lactose, mannitol, cornstarch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically acceptable and compatible carriers. Lozenge forms can comprise the active ingredient in a flavor, usually sucrose and acacia or tragacanth, as well as pastilles comprising the active ingredient in an inert base, such as gelatin and glycerin or sucrose and acacia emulsions, gels, and the like containing, in addition to the active ingredient, such carriers as are known in the art.

The therapeutic agent conjugated with a composition of this invention, alone or in combination with other suitable components, can be made into aerosol formulations to be administered via inhalation. These aerosol formulations can be placed into pressurized acceptable propellants, such as dichlorodifluoromethane, hydrofluorocarbon (such as HFC 134a and/or 227), nitrogen, and the like.

Formulations suitable for parenteral administration include aqueous and non-aqueous solutions, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried condition requiring only the addition of the sterile liquid carrier, for example, water, for injections, immediately prior to use. Extemporaneous injection solutions and suspensions can be prepared from sterile powders, granules, and tablets of the kind previously described.

The dose administered to an animal, particularly a human, in the context of the present invention should be sufficient to affect a therapeutic response in the animal over a reasonable time frame. The specific dose level and frequency of dosage may vary, depending upon a variety of factors, including the activity of the specific active compound, its metabolic stability and length of action, rate of excretion, mode and time of administration, the age, body weight, health condition, gender, diet, etc., of the subject, and the severity of, for example, the cancer. Any effective amount of the compound can be administered, e.g., from about 1 mg to about 500 mg per day, about 50 mg to about 150 mg per day, etc.

In another embodiment of this invention, the compositions can be used in a method of identifying and removing heavy metals or radioactive materials from a contaminated area or entity. The compositions, including an imaging tracer and a chelate complex, are used as scavengers and identifiers for heavy metal detoxification or nuclear decontamination. In this application, the ¹⁹F resonance frequency is used to identify metal ions of unknown identity. This has biomedical (e.g., heavy metal detoxification), environmental (e.g., waste water treatment) and defense (e.g., nuclear decontamination) applications. Fluorinated chelators can be used to scavenge metal ions (stable or radioactive) for heavy metal detoxification and/or nuclear decontamination. Moreover, the ¹⁹F resonance frequency will be shifted by the metal ion bound by the chelator ligand of the composition, and the resulting resonance frequency can be used to identify the metal ion. The identification can be done either in vivo or in vitro, using, for example, ¹⁹F MRI or ¹⁹F MRS.

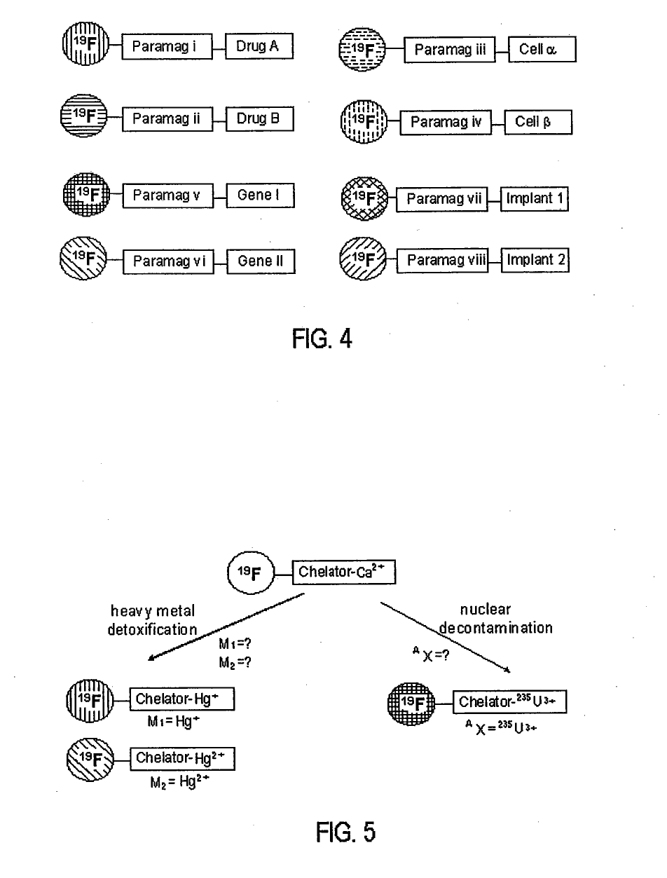

FIG. 5 generally illustrates the use of compositions according to this invention for identifying and removing undesirable metal ions from an animal or other environment. In FIG. 5, the composition including a ¹⁹F magnetic resonance imaging tracer conjugated with a chelate complex of a ligand combined with a calcium ion is shown removing mercury and uranium-235. The chelator is used to complex with metal ions for detoxification or decontamination while the ¹⁹F signal is used for identifying the metal ions removed. During the process, the calcium is replaced by the metal ions to be removed, as the undesirable metal ions form stronger complexes with the chelator. For in vivo uses, calcium is used in the initial chelate complex because calcium is harmless. Other ions can be used as well, such as magnesium. For in vitro use, the calcium can be omitted, or a broader range of ions can be used in the initial chelate complex.

Depending on the nature of the metal ion(s) removed, the ¹⁹F resonance frequency will change. The different resonances can be considered or represented by different colors, and form a basis for identification. Referring to the example in FIG. 5, as Hg⁺ is paramagnetic and Hg²⁺ is diamagnetic, the two ions will result in distinct ¹⁹F resonance frequencies, which are used to identify the presence of both mercury ions. As mentioned above, such identification can be performed in vivo or in vitro, such as using ¹⁹F magnetic resonance spectroscopy as the MR imaging technique.

The present invention is described in further detail in connection with the following examples which illustrate or simulate various aspects involved in the practice of the invention. It is to be understood that all changes that come within the spirit of the invention are desired to be protected and thus the invention is not to be construed as limited by these examples. It is to be understood that any discussion of theory is included to assist in the understanding of the subject invention and is in no way limiting to the invention in its broad application.

### EXAMPLES

As discussed above, clinical MRI is a mono-chromic technology that detects the ¹H2O signal, which has different intensities in different tissues, but all at the same frequency. Differences in the signal intensity result in black-and-white images. ¹H MRI is good at collecting anatomical and (patho)physiological information and hence is extremely valuable in disease diagnosis and therapy assessment. However, ¹H MRI is not well suited for tracking and quantifying objects inside the body, because the ¹H signal emitted by an object will be blurred and even drowned out by the background ¹H signals, particularly the ¹H2O signal. Non-invasive tracking and quantification of objects can be of immense value to biology and medicine. For example, if the metastasis of cancer cells and the differentiation of stem cells can be observed in the context of the whole body, a much better understanding of cancer and stem cell biology can be achieved. Monitoring the interactions between cancer cells and anti-cancer drugs in living subjects can provide new insight to cancer therapy. Tracking and quantifying drugs with narrow therapeutic indices enables physicians to tailor drug therapy for each patient.

For in vivo object tracking, ¹⁹F MRI has two principle advantages over ¹H MRI. First, unlike the ¹H signal, the ¹⁹F signal has no endogenous background. Second, while ¹H imaging agents are detected indirectly through the ¹H2O signal, ¹⁹F imaging agents are detected directly by MRI. Once an object is labeled by a ¹⁹F imaging agent, the ¹⁹F signal at that specific radiofrequency will come entirely from the labeled object.

The current MRI detection limit for a single ¹⁹F nucleus is about 10 mM. In one embodiment of this invention, this limit is improved significantly, for example, by 10⁴ fold to 1 μM. This increase can be accomplished by increasing the amount of fluorine atoms in the ¹⁹FIT molecule. As an example, in one embodiment of this invention, 100-200 fluorine atoms can be incorporated into a single ¹⁹FIT molecule in a spherically symmetric manner so that they will emit a single ¹⁹F signal. This increases the ¹⁹F signal intensity by 100-200 fold. Also, shortening the longitudinal relaxation time (T₁) of the ¹⁹F signal can increase the ¹⁹F signal intensity by allowing the collection of more signal transients within a given data acquisition time. T₁ of ¹⁹F can be shortened by paramagnetic ions. By shortening T₁ from 1 s to 100 μs, for example, the ¹⁹F signal intensity will increase by 100 fold. Together, these two measures can be used to lower the detection limit of the ¹⁹F signal by 10⁴ fold from 10 mM to 1 μM.

¹H MRI is not quantitative because the imaging agent is detected indirectly through the ¹H2O signal. A ¹H imaging agent emits no MRI signal. There is no simple relationship between the ¹H signal intensity and the imaging agent concentration. In contrast, a ¹⁹F imaging agent emits a MRI signal that is directly detected by ¹⁹F MRI. The ¹⁹F signal intensity is directly proportional to the imaging agent concentration. However, the ¹⁹F signal intensity is determined not just by the concentration, but also by the relaxation times, of the imaging agent, as shown by the following equation:

S=S₀·[1−exp(−TR/T₁)]·exp(−TE/T₂)

where S is the MRI signal intensity; S₀ is the spin density which is proportional to imaging agent concentration; and T₁ and T₂ are the longitudinal and transverse relaxation times, respectively. TR and TE are MRI experimental parameters. T₁ and T₂ vary from tissue to tissue and such tissue-dependent relaxation behavior is essential for ¹H MRI. However, for a quantitative ¹⁹F MRI technology, T₁ and T₂ of the ¹⁹F signal need to be tissue-independent so that the ¹⁹F signal intensity is solely determined by the concentration of the imaging agent.

Because electronic magnetic moments are much larger than nuclear magnetic moments, paramagnetic ions dominate the relaxation of nearby nuclear spins (C. Belle et al., ¹⁹F NMR: An underused efficient probe for paramagnetic metal centers in bioinorganic solution chemistry. Coord. Chem. Rev. 253, 963-976 (2009)). Using a paramagnetic ion as the magnetic signal modulator M in the imaging agent ¹⁹FIT-M according to one embodiment of this invention is believed to make the ¹⁹F T₁ and T₂ tissue-independent. With fixed T₁ and T₂ values, ¹⁹F signal intensity is determined solely by the concentration of the imaging agent. In summary, the paramagnetic ion M in ¹⁹FIT-M provides the following: shifts the ¹⁹F signal frequency to generate color; enhances the ¹⁹F signal intensity by reducing Tᵢ; dominates the ¹⁹F signal relaxation to make its T₁ and T₂ tissue-independent.

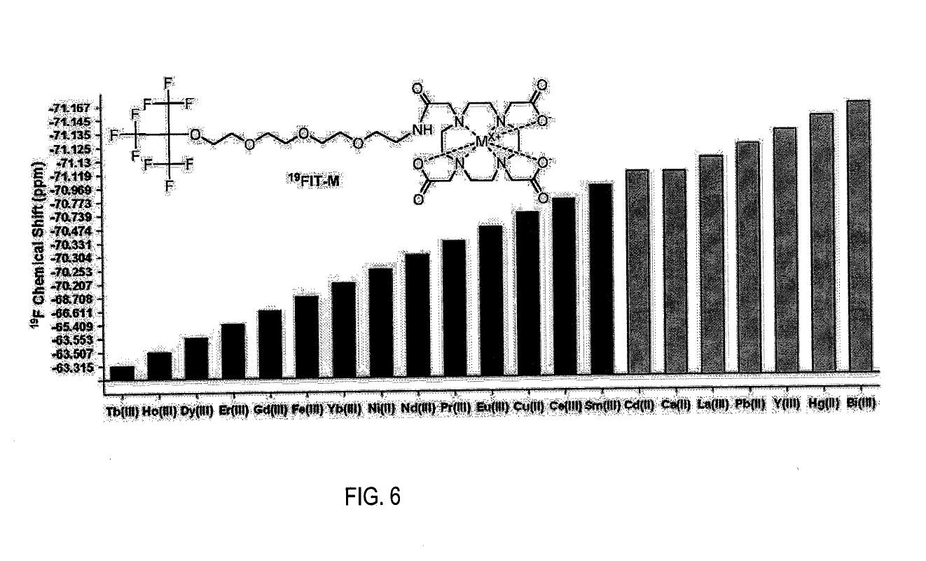

To demonstrate that the fluorinated paramagnetic complex imaging agents of this invention are promising imaging agents for multi-colored ¹⁹F MRI, the exemplary ¹⁹FIT-M shown in FIG. 6 was prepared, and had an aqueous solubility higher than 500 mM. The resonance frequency of the exemplary ¹⁹FIT-M including each of the ions listed in FIG. 6 was recorded. In FIG. 6, the darker bars indicate paramagnetic ion complexes and the lighter bars indicate diamagnetic complexes. As shown in the graph of FIG. 6, the different metal ions led to different ¹⁹F resonance frequencies, ranging from δ(¹⁹FIT-Bi³⁺)=−71.17 ppm to δ(¹⁹FIT-Tb³⁺)=−63.32 ppm. In each case, there was a single ¹⁹F signal.

The ¹⁹F T₁ and T₂ were determined under different solution conditions for the paramagnetic ¹⁹FIT-Gd³⁺ and for the diamagnetic ¹⁹FIT-Y³⁺ from the above example (and shown in FIG. 6). The conditions and results are summarized below in Table 1. The percentage numbers for Conditions 2-5 were calculated relative to T₁ or T₂ under Condition 1. As shown in Table 1, the paramagnetic Gd³⁺ drastically shortened T₁ and T₂; made T₁ essentially independent of its environment; and significantly reduced the environment-dependency of T₂. For example, the weakly paramagnetic O₂ had little effect on the ¹⁹F T₁ and T₂ of ¹⁹FIT-Gd³⁺.

The data in Table 1 show that the fluorinated paramagnetic complexes according to this invention are promising imaging agents for multi-colored ¹⁹F MRI. 19F T₁ and T₂ were also determined for ¹⁹FIT-Gd³⁺ and ¹⁹FIT-Y³⁺ at different pH values (pH 6 7 & 8). Relaxation times of neither complex showed significant pH dependency. The NMR field strength for the testing was 11.7 Tesla.

TABLE 1
Effect on the ¹⁹F relaxation times by paramagnetic Gd³⁺ and diamagnetic Y³⁺.

| | Relaxation | Condition | Condition | Condition | Condition | Condition |
|---|---|---|---|---|---|---|
| Sample | Times (ms) | 1 | 2 | 3 | 4 | 5 |
| ¹⁹FIT-Gd³⁺ | T₁ | 4.24 | 4.27 | 3.97 | 4.27 | 4.52 |
| (50 mM) | | | (−0.7%) | (−6.4%) | (−0.7%) | (6.6%) |
| | T₂ | 1.66 | 1.56 | 1.22 | 0.86 | 0.55 |
| | | | (−6.0%) | (−26.5%) | (−48.2%) | (−66.9%) |
| ¹⁹FIT-Y³⁺ | T₁ | 1498 | 1074 | 2133 | 1211 | 1200 |
| (50 mM) | | | (−28.3%) | (42.4%) | (−19.2%) | (−19.9%) |
| | T₂ | 1057 | 665 | 1511 | 214 | 139 |
| | | | (−37.1%) | (43.0%) | (−79.8%) | (−86.8%) |

Condition 1: PBS buffer saturated with N₂, 25° C., pH 7. (PBS: phosphate-buffered saline)
Condition 2: PBS buffer saturated with O₂, 25° C., pH 7.
Condition 3: PBS buffer saturated with N₂, 45° C., pH 7.
Condition 4: Human sera with 10% D₂O saturated with N₂, 25° C., pH 7.
Condition 5: 10% w/v bovine serum albumin in PBS buffer saturated with N₂, 25° C., pH 7.

Thus the invention provides a composition that allows for multi-chromic imaging. By providing different magnetic resonances using different paramagnetic or diamagnetic entities, the compositions allow for simultaneous tracking and/or identification of agents in the body. The compositions are also useful in identifying and removing unknown metals in a patient or other environment.

The invention illustratively disclosed herein suitably may be practiced in the absence of any element, part, step, component, or ingredient which is not specifically disclosed herein.

While in the foregoing detailed description this invention has been described in relation to certain preferred embodiments thereof, and many details have been set forth for purposes of illustration, it will be apparent to those skilled in the art that the invention is susceptible to additional embodiments and that certain of the details described herein can be varied considerably without departing from the basic principles of the invention.

1. A composition comprising a magnetic resonance (MR) imaging tracer conjugated with at least one of a paramagnetic functional group or a chelate complex of a ligand combined with a paramagnetic ion or a diamagnetic ion.

2. The composition according to claim 1, wherein the MR imaging tracer comprises fluorine or phosphorus.

3. The imaging agent according to claim 1, wherein the MR imaging tracer is detectable by ¹⁹F MRI or ¹⁹F MRS.

4. The composition according to claim 1, wherein the paramagnetic functional group is nitroxide.

5. The imaging agent according to claim 1, wherein the paramagnetic ion is selected from the group consisting of Cu²⁺, Ni²⁺, Fe³⁺, Eu³⁺, Gd³⁺, Tb³⁺, and combinations thereof.

6. The composition according to claim 1, wherein the ligand comprises 1,4,7,10-tetraazacyclododecane-1,4,7,10-tetraacetic acid (DOTA) or derivatives thereof.

7. The composition according to claim 1, wherein the MR imaging tracer is conjugated to a plurality of the at least one of a paramagnetic functional group or the chelate complex, the MR imaging tracer comprises a branching module including a plurality of branching units, and each of the branching units is conjugated to one of at least one of a paramagnetic functional group or the chelate complex

8. The composition according to claim 1, comprising the structure: 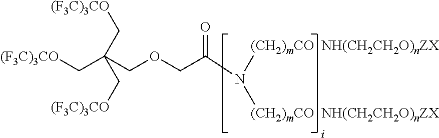
wherein each X is independently the paramagnetic functional group, the chelate complex or an ¹H contrast agent, and at least one X is the paramagnetic functional group or the chelate complex; each Z is independently a linker group or bond; m is independently a positive integer; n is independently zero or a positive integer; and i is zero or a positive integer.

9. The composition according to claim 8, comprising the structure:

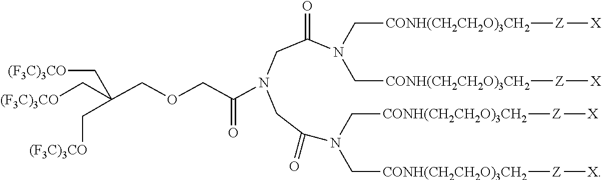

10. The composition according to claim 1, comprising the structure: 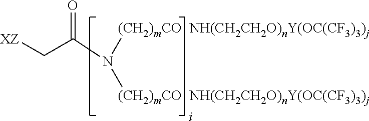
wherein X includes the paramagnetic functional group, the chelate complex or an ¹H contrast agent; each Y and Z is independently a linker group or bond; m is independently a positive integer; j is independently a positive integer; n is independently zero or a positive integer; and i is zero or a positive integer.

11. The composition according to claim 1, comprising the structure: 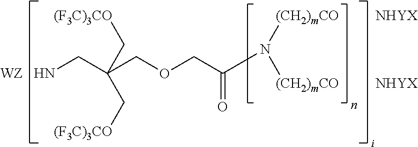
wherein each X or W includes the paramagnetic functional group, the chelate complex or an ¹H contrast agent, and at least one X or W includes the paramagnetic functional group or the chelate complex; each Y and Z is independently a linker group or bond; m is independently a positive integer; and n and i are independently positive integers.

12. The composition according to claim 8, wherein each Y or Z are selected from a group consisting of an amide bond, an ester bond, a heterocyclic, or a triazole ring.

13. The composition according to claim 1, wherein the MR imaging tracer comprises a dual-nuclei (e.g., ¹H-¹⁹F) imaging agent.

14. The composition according to claim 1, wherein the magnetic resonance imaging tracer is a first magnetic resonance (MR) imaging tracer conjugated with at least one of the paramagnetic functional group or the chelate complex, and further comprising a second magnetic resonance (MR) imaging tracer conjugated with at least one of a paramagnetic functional group or a chelate complex of a ligand combined with a second magnetic signal modulator, wherein the second MR imaging tracer is the same or different from the first MR imaging tracer, and the second magnetic signal modulator is different that the magnetic signal modulator.

15. The composition of claim 1, further comprising a therapeutic agent joined to the composition.

16. The imaging agent of any of claim 15, wherein the therapeutic agent is selected from the group consisting of a drug, a prodrug, a gene, a cell, and an implant.

17. A pharmaceutical composition comprising the composition of claim 1, and one or more pharmaceutically acceptable carriers.

18. A method of generating a magnetic resonance image, the method comprising:
administering to an animal a dose of the composition according to claim 1; and
measuring an amount of the composition in a tissue or organ of the animal using magnetic resonance imaging (MRI).

19. The method according to claim 18, further comprising:
administering to the animal a dose of a second composition including a second magnetic resonance (MR) imaging tracer conjugated with at least one of a second paramagnetic functional group or a chelate complex of a ligand combined with a second magnetic signal modulator; and
measuring an amount of the second composition in a tissue or organ of the animal using MRI; and
creating an image based upon the measurement of the composition and the second composition.

20. A method of removing and identifying heavy metals or radioactive materials from a contaminated area or entity, the method comprising administering a magnetic resonance (MR) imaging tracer conjugated with a chelate complex of a ligand combined with a calcium ion or a magnesium ion to the contaminated area or entity, wherein the calcium ion or magnesium ion is replaced by metal ions from the contaminated area or entity.
